(19) United States
(12) Patent Application Publication
(10) Pub. No.: US 2026/0265202 A1
(43) Pub. Date: Sep. 10, 2026

(54) N-(N-AMINOALKYL)PHENANTHRIDIUNIUM SERIES OF CHLORIDE-SENSITIVE FLUOROPHORES AND METHODS OF USE THEREOF

(71) Applicant: The General Hospital Corporation, Boston, MA (US)

(72) Inventors: Kieran P. Normoyle, Cambridge, MA (US); Kevin Staley, Boston, MA (US)

(21) Appl. No.: 19/128,124

(22) PCT Filed: Nov. 7, 2023
(86) PCT No.: PCT/US2023/078984

§ 371 Date: May 7, 2025

Related U.S. Application Data
(60) Provisional application No. 63/423,699, filed on Nov. 8, 2022.

(51) Int. Cl. C07D 221/12

(52) U.S. Cl. CPC C07D 221/12

(57) ABSTRACT

Disclosed herein are compounds which are chloride-sensitive fluorophores and methods of manufacture and of use thereof.

### CROSS-REFERENCE TO RELATED APPLICATIONS

This application is the U.S. National Phase Application under 35 U.S.C. § 371 of International Patent Application No. PCT/US2023/078984, filed on Nov. 7, 2023, which claims the benefit of U.S. Provisional Patent Application Ser. No. 63/423,699, filed on Nov. 8, 2022, the entire contents of each are hereby incorporated by reference.

### STATEMENT OF FEDERALLY SPONSORED RESEARCH

This invention was made with government support under Grant No. W81XWH-15-2-0069 awarded by the Defense Health Agency, Medical Research and Development Branch, and NS116852 awarded by the National Institutes of Health. The government has certain rights in the invention.

### FIELD

This disclosure relates to compounds which are chloride-sensitive fluorophores and methods of manufacture and of use thereof.

### BACKGROUND

The clinical implications of finding new drug targets fundamental to neuronal function are immediately applicable to the field of epilepsy and will be of interest to neurologists in general. Several prevalent diseases are associated with epilepsy, from Alzheimer Disease to autism, and epilepsy is a common complication of stroke.

Epilepsy is a life-altering condition in which more than 30% of cases are medically intractable. Both established and newly available anti-seizure medications have similar rates of failure, and patients who do not respond to first line treatment are unlikely to respond at all. For all the progress that has been made in drug discovery and development there is still great clinical need for new drugs targeting the core mechanisms that cause epilepsy.

The brain is often thought of as a biological circuit board containing neurons, the cells which use the cross-membrane flow of charged electrolytes like sodium, calcium, and chloride to conduct electricity and/or regulate this conduction. Neurons are responsive to their environment, and that environment is changed in response to ischemic or traumatic injury that leads to epilepsy.

Chloride (Cl⁻) is the principal negatively charged electrolyte (anion) that flows across the semi-permeable cell membrane of neurons to regulate their physiological electrical activity. The electrical effect of chloride is to polarize the neuron and prevent the activation of electrical signals. This limitation of electrical firing to only those times in which such activity is appropriate underlies the proper function of the brain and nervous system. When this system malfunctions, neurons and their targets (other neurons, muscles, etc.) inappropriately send and receive electrical signals, leading to seizure. When this becomes a chronic condition, it is termed epilepsy. Epilepsy affects 3.4 million Americans, and 1 in 26 will develop some form of epilepsy at some point in their life. Despite many types and classes of anti-seizure medications, approximately one third of patients are still in need of clinically effective treatment.

Canonically, Cl⁻ flows into neurons when Cl⁻ channels are activated by the inhibitory neurotransmitters GABA and/or glycine. This makes it harder for neurons to discharge an electrical signal, i.e. to ‘fire.’ The role of proper handling of Cl⁻, then, is to allow the proper regulation of neurons whereas seizures represent the breakdown of this regulation resulting in improper firing of neurons. Anti-seizure drugs come in many types, but they generally have in common that they affect a neurotransmitter receptor spanning the neuronal membrane. However, if these receptors were truly the fundamental determinant of how neurons regulate themselves then it would not be the case that nearly one-third of epilepsy patients still are in need of an effective medication. The field is missing something fundamental about the pathophysiology of seizures, and understanding how to target the relevant processes and pathways is critical to develop different and more effective treatments.

To monitor changes to the neuronal environment, i.e., changes to the extracellular matrix surrounding the neuron, and the pathophysiology of seizures, chloride ions (Cl⁻) need to be measured both inside and outside the neuron in a non-invasive way. Intracellular chloride can be measured with chloride-sensitive fluorescent proteins or a number of commercially available chloride-sensitive fluorescent small molecules. The small molecule experiences a quenching of their fluorescence proportional to the concentration of chloride they encounter. Importantly, these are optimized for typical concentrations of chloride ([Cl⁻]) that one would expect to find inside cells ranging from 5-20 millimolar (mM). In contrast, extracellular chloride concentration ([Cl⁻]ₒ) is an order of magnitude higher than this, exceeding the dynamic range of currently available chloride-sensitive small molecules and creating a technological gap in which measurement of [Cl⁻]ₒ has relied on invasive methods that has made accurate measurements of [Cl⁻]ₒ difficult to obtain despite the clear need to do so.

### SUMMARY

The present disclosure relates to novel chloride-sensitive fluorophore compositions comprising of a tricyclic phenanthridine base first structure and an alkyl amine second structure appended to nitrogen of the phenanthridine ring as illustrated in FIGS. 1 and 2. In some embodiments, the disclosure relates to compositions thereof.

In some embodiments, the disclosure relates to a method for non-invasively measuring the concentration of chloride in the extracellular space comprised of administrating compositions disclosed herein to a cell, tissue or organism and wherein the fluorescence is indicative of said extracellular chloride concentration.

In some embodiments, the disclosure relates a general method for rapidly measuring salinity in biological, medical, food production or non-biological systems comprising of the application of one or more of the chloride-sensitive fluorophore compounds herein, and wherein the fluorescence is indicative of the amount of salinity in said system.

Some embodiments provide a conjugate of formula F-L-P, wherein F is a heteroaryl, L is a linker, and P is a cap group or a polymer chosen from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin, and carrageenan, or a derivative of any of the foregoing.

Some embodiments provide a conjugate of formula (Ia)

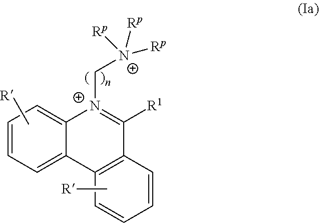

wherein each R′ and R¹ is independently chosen from hydrogen, halogen, alkyl, and heteroalkyl, n is chosen from 1, 2, 3, 4, 5, and 6, and each R^{p} independently chosen from alkyl, heteroalkyl, benzyl, and aryl. Some embodiments provide a conjugate of formula (Ib)

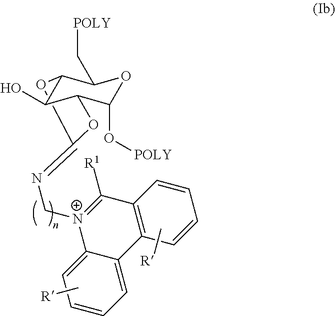

wherein each R′ and R¹ is independently chosen from hydrogen, halogen, alkyl, and heteroalkyl, n is chosen from 1, 2, 3, 4, 5, and 6, and each POLY is independently chosen from a dextran polymer.

In some embodiments, the disclosure relates a method for measuring the extracellular chloride concentration in an extracellular space, comprising administrating a conjugate disclosed herein a cell, tissue or organism and wherein the fluorescence of the compound indicates the extracellular chloride concentration.

### DESCRIPTION OF THE DRAWINGS

FIG. 1. Schematic diagram of Optimization of Extracellular Chloride Measurement. Candidate probes based on quiniline, isoquiniline, and phenanthridine, were developed. Phenanthridine was chosen due to its longer wavelength (lower energy) excitation maximum and increased brightness in our system. Further, the distance between the two nitrogen atoms in the molecule was inversely proportional to both the chloride sensitivity and the brightness of the fluorescence probe. Finally, the trimethyl ‘cap’ added to eliminate pH-sensitivity could be replaced with a primary amine amenable to conjugation to a large carrier molecule such as dextran. Insensitivity to pH would be preserved in the physiological range, and the resulting conjugated compound would be restricted to extracellular space.

FIG. 2. depicts a scheme for the synthesis of N-(4-aminobutyl)phenanthridinium (ABP) and optional Conjugation to Dextran.

FIG. 3 ABP-Dextran Specificity for Extracellular Space and Calibration. A ABP intensity image of CA1 pyramidal and associated radial layers demonstrating ABP-dextran exclusion from cell bodies. B Stem-Volmer calibration plot yielding linear relationship between ABP-Dextran fluorescence lifetime and [Cl⁻](plotted as [tₘₐₓᵢₘᵤₘ/t_{[Cl—]}] vs [Cl—]; please see text for further details on Stem-Volmer plots). C ABP lifetime image of CA1 pyramidal and associated radial layers demonstrating 1) lifetimes within approximate calibrated range of ABP; 2) exclusion from cell bodies and large collateral fibers; 3) longer lifetimes (cooler colors) around and between pyramidal cell bodies in pyramidal cell layer (PCL, white dashed lines) indicating lower [Cl⁻]ₒ.

FIG. 4 depicts Limitations of existing chloride-sensitive fluorophores. Commercially available chloride-sensitive fluorophores were developed to measure intracellular concentrations of chloride (Cli), which are typically an order of magnitude less than extracellular chloride concentrations (Clo). A) Sample data generated from published parameters of several chloride-sensitive probes and their published kSV values (see legend), each normalized for co-plotting, with the notable addition of our novel sensor ABP-dextran and empirical kSV. The most suitable Clo sensors will have a large part of their total lifetime shortened, and emission quenched, as Clo increases from physiological Cli values to typical CSF values (i.e., 40-110 mM; shaded region). B) Three ‘generations’ of heterocyclic addition chemistry applied to chloride measurement. The addition of sultones, bromated alkyl chains, and bromated alkylamines give rise to commercially available chloride sensors (SPQ and MEQ, respectively) as well as APQ, the most closely related Verkman series compound to ABP. Each example compound here has a (6-methoxy)quinoline base. C) Several parameters were tuned in order to fashion a chloride sensor that met each of our stated criteria. The alkyl chain length was increased to increase kSV, but at the expense of overall brightness (1); a more complex heterocyclic structure was employed to red-shift excitation out of spectral overlap with autofluorescent species, taking care not to red-shift emission past the blue spectrum (2); the trimethylated terminal nitrogen was altered to allow conjugation to dextran and resultant extracellular sequestration (3). D) Focusing on chloride concentrations greater than 40 mM up to typical CSF chloride of 110 mM (shaded region in A), the linear slope of each compound is calculated and compared. SPA and NBSQ are improved by this metric but suffer from unacceptably short excitation wavelength and long emission wavelength, respectively.

FIG. 5 depicts the characterization of novel chloride-sensitive probe ABP and its conjugation to dextran. ABP was synthesized as a primary amine amenable to dextran conjugation (see FIG. 2). A) Fundamental optical properties of ABP demonstrate a bimodal absorbance peak (blue line) with similar fluorescence excitation throughout (black line). Fluorescence emission data was gathered at 360 nm excitation and demonstrated a peak at approximately 420 nm. B) Two-photon excitation (2PE) of ABP-dextran at given wavelengths through a 445/58 emission filter. Note that optical data generated from ABP conjugated to 10KDAa dextran (ABP-dextran) is shown; properties of unconjugated ABP were not appreciably different. C) Characteristics of ABP-Dextran as a function of molar ratio of primary amine ABP reagent to 10KDAa activated dextran. Pairs of bars are centered at molar ratios of 2.4, 8, and 24 ABP:Dextran; calculated molar ratios achieved (‘final’) are annotated above pairs of bars. Intensity at lowest molar ratio of ABP added to dextran is significantly attenuated (2.4× vs 8× p=0.032; 2.4× vs 24× p=0.005), but greater molar ratios of ABP to dextran failed to significantly impact intensity readings (8× vs 24× p=0.220) consistent with all available conjugation sites being occupied. As expected, ABP-dextran lifetime did not change with increased ABP loading because fluorescence lifetime is not dependent on fluorophore concentration and Cl⁻ was held constant (p-value range: 0.5765>p>0.7538). D) Calibrations of each ABP-dextran studied in (C) were carried out to obtain Stem-Volmer constants (kSV) for each, a measure of chloride sensitivity (see Methods). kSV did not change appreciably among dextrans with differing ABP label ratios (left bar; mean+/−SD: 15.7+/−0.8; p=0.0) nor was kSV significantly different from later, fully-optimized ABP-dextran batches (right bar; mean+/−SD: 14.7+/−0.5). C-D: mean+/−SD of lifetime or intensity data fit to normal distribution, i.e. mean+/−sigma(m+/−s), p-values reflect two-sample t-tests.

FIG. 6 explains the optimization of dextran-conjugated ABP. Once ABP-dextran synthesis and conjugation was optimized, the concentration of this sensor was varied to optimize experimental conditions. A) Intensity, but not lifetime, increases linearly with ABP-dextran concentration from 100 mg/mL to 1000 mg/mL (10-100 mM). Note that as concentration of ABP-dextran increases, ABP lifetime variance decreases while mean lifetime changes only slightly. B) Intensity histograms of three representative concentrations of ABP-dextran demonstrating that as ABP-dextran increases, both brightness and variance are increased resulting in broader peaks of reduced amplitude. C) Lifetime histograms comparing ABP-dextran concentrations corresponding to the data in (B) demonstrating that as ABP-dextran concentration increases variance decreases. This creates narrower distributions of lifetimes with greater peak pixel counts as ABP-dextran concentration increases, even as intensity distribution broadens (B) over the same half log-step of ABP-dextran concentration shown in B&C. This narrowing of lifetime distribution is asymmetrical, with longer lifetimes reduced more than shorter lifetimes as ABP-dextran concentration increases, resulting in the mean lifetime value drifting lower with increasing ABP-dextran (11.2% reduction over a 10-fold increase in concentration; A, blue line) while the mode lifetime value changes <1% from 50 mM to 100 mM ABP-dextran (blue vs green) and only 5% for the half log-step data range shown. D) Coefficient of Variation decreases with increasing ABP-dextran concentration, most notably for lifetime values. A: mean+/−SD of lifetime or intensity data fit to normal distribution, i.e. mean+/−sigma(m+/−s); β-C: example histograms of raw data; D: coefficient of variation (SD/mean) of the data displayed in A.

FIG. 7 explains the reproducibility and performance of ABP-dextran. A) pH independence of ABP-dextran over the physiological concentration range (6.8-7.8 pH). B) Bubbling with 95% O₂/5% CO₂ causes a statistically significant reduction in lifetime of less than 4% of unquenched signal. This stable level of quenching is experimentally acceptable as long as calibration solutions are also bubbled with 95% O₂/5% CO₂. C) Absence of ABP-dextran induced quenching in the presence of other biologically relevant anions: phosphate, methyl sulfonate, bicarbonate and gluconate. D) ABP-dextran performs similarly before and after autoclave, allowing long-term in vivo experiments. E) Optimized synthetic and experimental procedures give reproducible results batch to batch; consecutive batches of ABP-dextran shown. F) Though separate FLIM-capable microscopes report differences in k^{SV} due to hardware-associated discrepancies, when raw values of each rig are normalized there is no significant difference between the calculated k^{SV} values, i.e. there is no difference in Cl⁻ sensitivity. A: mean+/−SD, no statistically significant differences; B: mean+/−SD, unpaired t-test p 0.0327; C-E: mean+/−SD of normal fit to data at each [Cl⁻]; F: mean+/−SD, unpaired t-test p 0.8884.

FIG. 8 depicts FLIM images from two separate microscopes are composites of intensity and lifetime components, where color encodes fluorescence lifetime. A) Murine hippocampal organotypic slice culture (DIV8) perfused with 500 mg/mL ABP-dextran in aCSF, CA1 and stratum radiatum regions shown (top-left versus bottom-right, respectively). This image was taken with an older rig equipped with Becker and Hickel (B&H, GmbH) FLIM hardware and software. B) Murine in vivo image taken in a second, specially equipped custom rig with custom FLIM hardware and Vidrio acquisition and FLIMJ analysis software. Layer 2/3 neocortex is shown as viewed through a cortical window placed at P27, at a depth of 178 mm from the overlying cortical surface. Large shadows are the result of blood vessels superficial to the field of view while small silhouettes are somatic shadows, each visible because of ABP-dextran exclusion from intraluminal and intracellular space, respectively.

### DETAILED DESCRIPTION

### Compositions

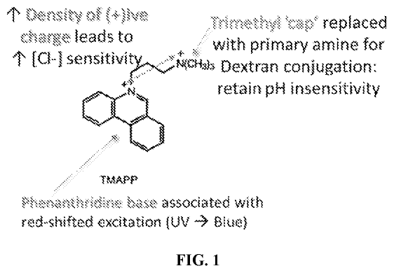

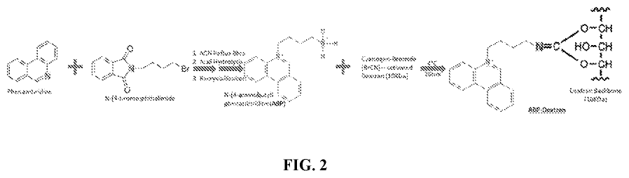

In some embodiments, the disclosure provides a novel chloride-sensitive fluorophore composition comprised of a tricyclic phenanthridine base first structure and an alkyl amine second structure appended to nitrogen of the phenanthridine ring as illustrated in FIGS. 1 and 2 of the Specification.

In some embodiments, the distance between the phenanthridine nitrogen and the amine group alkyl chain may be 3 to 5 carbons.

In some embodiments, the distance between the phenanthridine nitrogen and the amine group alkyl chain is 4 carbons.

In some embodiments, a quaternary amine, covalently bonded to three alkyl chains, as illustrated in FIG. 2 of the Specification, is substituted for the primary amine in the second structure.

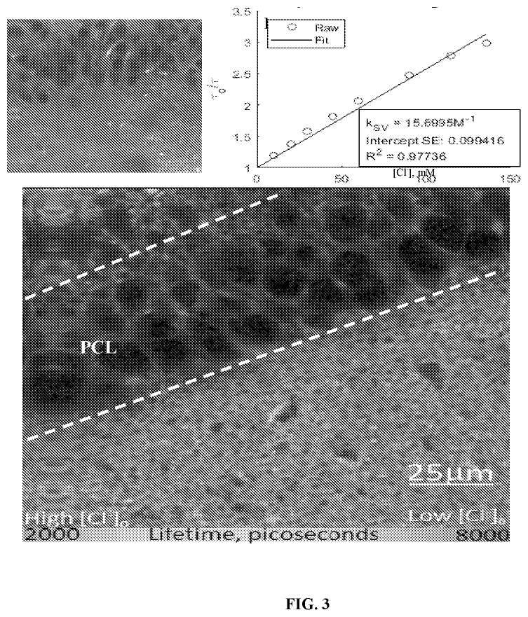

In some embodiments, the alkyl amine group is conjugated to a large bio-compatible substance, as illustrated in FIG. 3 of the Specification, to keep said fluorophore compound excluded from the intracellular space.

In some embodiments, the large bio-compatible substance is dextran.

Some embodiments provide a conjugate of formula F-L-P, wherein F is a heteroaryl, L is a linker, and P is a cap group or a polymer chosen from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative of any of the foregoing.

In some embodiments, the heteroaryl is a fused bicyclic or a fused tricyclic heteroaryl. In some embodiments, the heterocycle is a fused bicyclic heteroaryl. In some embodiments, the heteroaryl is a fused tricyclic heteroaryl. In some embodiments, the heteroaryl comprises at least one nitrogen atom. In some embodiments, the heteroaryl has one nitrogen atom. In some embodiments, the heteroaryl is chosen from indole, isoindole, indolizine, benzofuran, isobenzofuran, benzothiophene, isobenzothiophene, indazole, quinoline, isoquinoline, quinolizine, purine, phthalazine, pteridine, naphthyridine, quinoxaline, quinazoline, cinnoline, benzoxazole, benzothiazole, benzimidazole, chromene, benzoxepine, benzoxazepine, benzoxadiazepine, benzothiepine, benzothiazepine, benzothiadiazepine, benzazepine, benzodiazepine, benzofurazan, benzothiadiazole, benzotriazole, carbazole, beta-carboline, acridine, phenazine, dibenzofuran, xanthene, dibenzothiophene, phenothiazine, phenoxazine, phenoxathiin, thianthrene, phenanthridine, phenanthroline, and perimidine. In some embodiments, the heterocycle is quinoline, phenanthridine, or acridine.

In some embodiments, wherein the linker is an alkylene or a heteroalkylene. In some embodiments, wherein the linker is an alkylene. In some embodiments, the linker is methylene, ethylene, propylene, butylene, or pentalene. In some embodiments, wherein the linker is a heteroalkylene.

In some embodiments, wherein P is N(R^{p})3+, wherein R^{p} is alkyl. In some embodiments, R^{p} is methyl.

In some embodiments, P is a polymer chosen from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative of any of the foregoing.

In some embodiments, P is dextran.

In some embodiments, P is mannan.

In some embodiments, P is pullulan.

In some embodiments, P is hyaluronic acid.

In some embodiments, P is hydroxyethyl starch.

In some embodiments, P is chondroitin sulphate.

In some embodiments, P is heparin.

In some embodiments, P is heparin sulphate.

In some embodiments, P is polyalkylene glycol.

In some embodiments, P is Ficoll.

In some embodiments, P is polyvinyl alcohol.

In some embodiments, P is amylose.

In some embodiments, P is amylopectin.

In some embodiments, P is chitosan.

In some embodiments, P is cyclodextrin.

In some embodiments, P is pectin.

In some embodiments, P is carrageenan.

In some embodiments, P has a molecular weight of 1 kDa to 5000 kDa molecular weight. In some embodiments, P has a molecular weight of 1,000 kDa to 2,000 kDa. In some embodiments, P has a molecular weight of 500 kDa to 1000 kDa In some embodiments, P has a molecular weight of 100 kDa to 1000 kDa. In some embodiments, P has a molecular weight of 100 kDa to 500 kDa. In some embodiments, P has a molecular weight of 50 kDa to 100 kDa. In some embodiments, P has a molecular weight of 10 kDa to 100 kDa. In some embodiments, P has a molecular weight of 10 kDa to 50 kDa. In some embodiments, P has a molecular weight of 25 kDa to 50 kDa. In some embodiments, P has a molecular weight of 5 kDa to 25 kDa. In some embodiments, P has a molecular weight of 1 kDa to 10 kDa. In some embodiments, P has a molecular weight of 5 kDa to 20 kDa. In some embodiments, P has a molecular weight of 5 kDa to 15 kDa. In some embodiments, P has a molecular weight of 5 kDa to 12 kDa. In some embodiments, P has a molecular weight of 5 kDa to 10 kDa. In some embodiments, P has a molecular weight of 1 kDa to 5 kDa. In some embodiments, P has a molecular weight of 10 kDa. In some embodiments, wherein P is dextran.

In some embodiments, the disclosure provides a conjugate of formula (Ia)

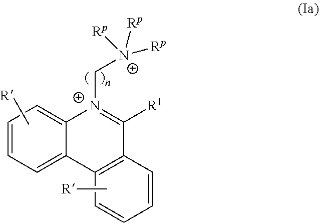

wherein each R′ and R¹ is chosen from hydrogen, halogen, alkyl, and heteroalkyl, n is chosen from 1, 2, 3, 4, 5, and 6, and each R^{p} independently chosen from alkyl, heteroalkyl, benzyl, and aryl.

In some embodiments, each R′ is hydrogen. In some embodiments, R¹ is hydrogen. In some embodiments, n is 3. In some embodiments, n is 4. In some embodiments, n is 5. In some embodiments, each R^{p} is alkyl. In some embodiments, each R^{p} is methyl.

In some embodiments, the disclosure provides a conjugate of formula (Ib)

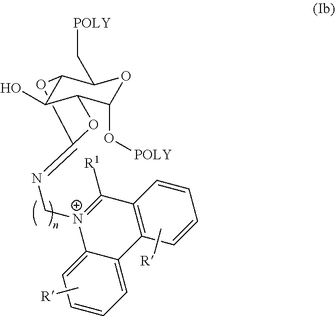

wherein each R′ and R¹ is chosen from hydrogen, halogen, alkyl, and heteroalkyl, n is chosen from 1, 2, 3, 4, 5, and 6, and each POLY is independently chosen from a dextran polymer.

In some embodiments, each R′ is hydrogen. In some embodiments, R¹ is hydrogen. In some embodiments, n is 3. In some embodiments, n is 4. In some embodiments, n is 5.

In some embodiments, the conjugate is a chloride-sensitive fluorophore.

In some embodiments, the disclosure provides compositions comprising the compounds or conjugates disclosed herein and pharmaceutically acceptable excipient.

In some embodiments, the compositions described herein are administered to a subject preferably by injection administration or infusion instillation. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrastemal injection and infusion. In preferred embodiments, the compositions for use in the methods described herein are administered by intravenous infusion or injection. The phrases “parenteral administration” and “administered parenterally” as used herein, refer to modes of administration other than enteral and topical administration, usually by injection. The phrases “systemic administration,” “administered systemically,” “peripheral administration,” and “administered peripherally” as used herein refer to the administration of an agent other than directly into a target site, tissue, or organ, such that it enters the subject's circulatory system and, thus, is subject to systemic metabolism and other like processes.

### Methods

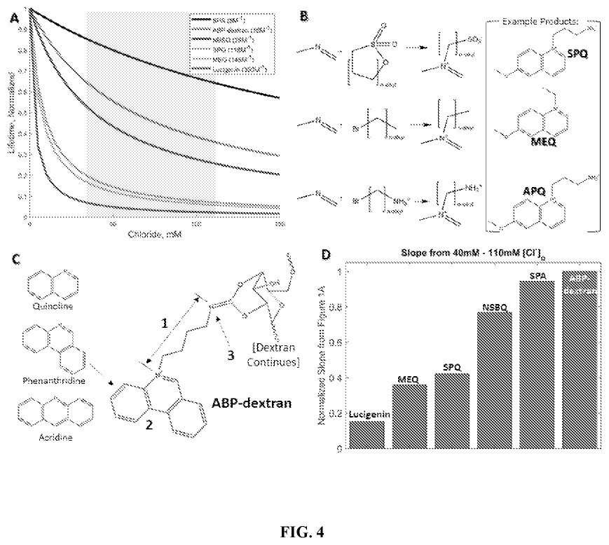

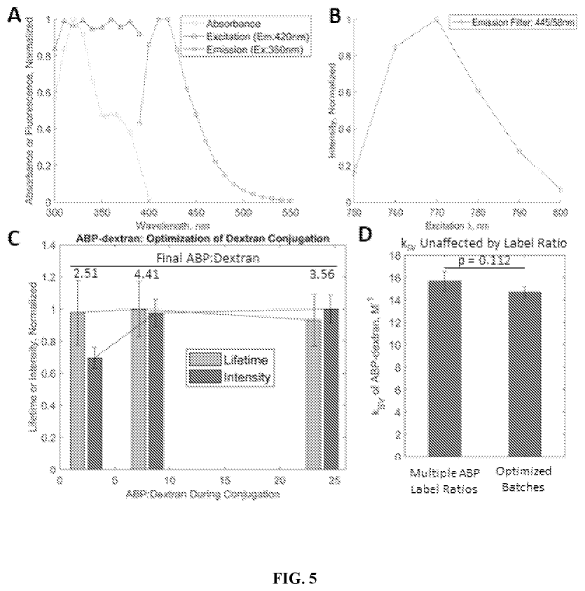

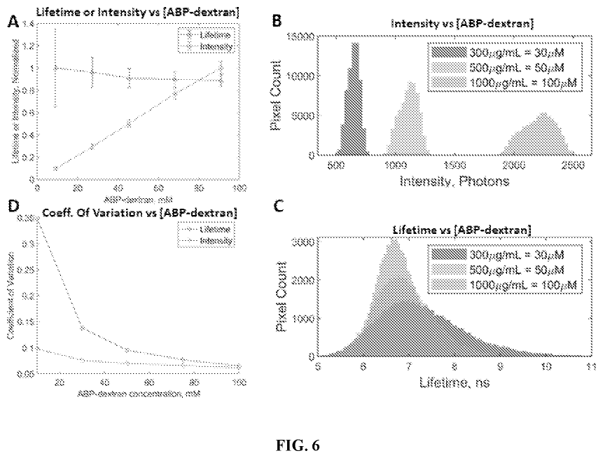

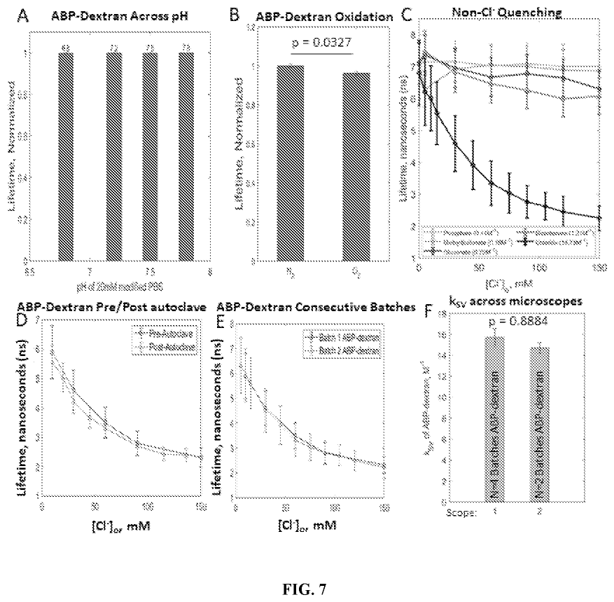

Previous extracellular Cl measurements typically rely on Cl-selective electrodes with tip diameters of 3-5 microns which causes a stab wound that is likely to disrupt local extracellular matrix in intact tissue and is inherently invasive. The fluorescent Cl-sensitive probe described here enables the first truly non-invasive measurements of Clo in vivo. The key features of ABP as a useful probe of Clo included: minimization of autofluorescence artifact by virtue of sufficient fluorophore brightness (FIG. 4 B) as well as excitation and emission spectra relative to autofluorescence (FIG. 5); appropriate sensitivity to chloride concentrations found in the extracellular space (FIG. 4); selective quenching of ABP by chloride vs other anions present in the extracellular space (FIG. 7 C); excitation as sufficiently long wavelengths so as to avoid phototoxicity (FIG. 5); and absence of toxicity (FIG. 6).

In some embodiments, the disclosure provides a method for non-invasively measuring the concentration of chloride in the extracellular space comprised of administrating one of the compositions in claim 4 or 5 to a cell, tissue or organism and wherein the fluorescence is indicative of said extracellular chloride concentration. In some embodiments, the non-invasive measurement of chloride in the extracellular space is used to determine scarring. In some embodiments, the non-invasive measurement of chloride in the extracellular space is used to determine the risk of scarring. In some embodiments, the non-invasive measurement of chloride in the extracellular space is used to monitor the effectiveness of a treatment for scarring, e.g., monitoring the reduction in the severity or amount of scarring over time.

In some embodiments, scarring occurs in tissues or organs of the body, such as the skin. In some embodiments, the non-invasive measurement of chloride in the extracellular space is used to determine glial scarring and used as a biomarker of the effectiveness of agents to reduce said scarring. In some embodiments, the non-invasive measurement of chloride in the extracellular space is used to diagnose glial scarring as a result of central nervous system (CNS) injury and as a biomarker of the effectiveness of agents to reduce said scarring. In some embodiments, glial scarring occurs in the brain. In some embodiments, glial scarring occurs in other tissues of the CNS (such as, but not limited to, the spinal cord). In some embodiments, the non-invasive measurement of chloride in the extracellular space, in conjunction with other biological measurements including intracellular chloride, can provide a more accurate indication of neuronal excitability. In some embodiments, the neuronal excitability is chronic. In some embodiments, the chronic neuronal excitability is associated with epilepsy.

In some embodiments, the disclosure provides a general method for rapidly measuring salinity in biological, medical, food production or non-biological systems comprised of the application of one or more of the chloride-sensitive fluorophore compounds disclosed herein and wherein the fluorescence is indicative of the amount of salinity in said system.

Some embodiments provide a method for measuring extracellular chloride concentration in a subject in need thereof, comprising administering a conjugate or compound disclosed herein or a pharmaceutical composition disclosed herein to the subject and measuring the fluorescence of the conjugate or compound. Some embodiments provide a method for measuring extracellular chloride concentration in a subject in need thereof comprising the step of administering a conjugate or compound disclosed herein or a pharmaceutical composition disclosed herein to the subject. Some embodiments provide a method for measuring extracellular chloride concentration in a subject in need thereof comprising the step of measuring the fluorescence of the conjugate or compound.

Some embodiments provide a method for measuring the extracellular chloride concentration in an extracellular space, comprising administering a conjugate or compound disclosed herein or a pharmaceutical composition disclosed herein to a cell, tissue or organism and wherein the fluorescence of the compound indicates the extracellular chloride concentration. In some embodiments, the measurement of the extracellular chloride concentration provides for a diagnosis of glial scarring. In some embodiments, the glial scarring is a result of CNS injury. In some embodiments, the measurement of the extracellular chloride concentration is used as a biomarker of the effectiveness of agents to reduce the scarring. In some embodiments, the measurement of the extracellular chloride concentration can provide a more accurate indication of neuronal excitability. In some embodiments, the measurement of the extracellular chloride is used in conjunction with other biological measurements. In some embodiments, the neuronal excitability is chronic. In some embodiments, the chronic neuronal excitability is associated with epilepsy. In some embodiments, the method is non-invasive.

Some embodiments provide a method for measuring extracellular chloride concentration in a sample from a subject, comprising contacting the sample with a conjugate or compound disclosed herein and measuring the fluorescence of the conjugate or compound.

Some embodiments provide a method for measuring extracellular chloride concentration in a sample from a subject, comprising the step of contacting the sample with a conjugate or compound disclosed herein. Some embodiments provide a method for measuring extracellular chloride concentration in a sample from a subject, comprising the step of measuring the fluorescence of the conjugate or compound.

In some embodiments, the subject is suspected of having a CNS injury. In some embodiments, the subject has been previously determined to have a CNS injury. In some embodiments, the subject is at risk of having a CNS injury. In some embodiments, the subject has a clinical record indicating a diagnosis of a CNS injury. In some embodiments, the CNS injury comprises glial scarring. In some embodiments, the CNS injury is glial scarring.

In some embodiments, the subject is suspected of having epilepsy. In some embodiments, the subject has been previously determined to have epilepsy. In some embodiments, the subject is at risk of having epilepsy. In some embodiments, the subject has a clinical record indicating a diagnosis of epilepsy. In some embodiments, the subject epilepsy.

The clinical implications of finding new drug targets fundamental to neuronal function are immediately applicable to the field of epilepsy and will be of interest to neurologists in general. Several prevalent diseases are associated with epilepsy, from Alzheimer Disease to autism, and epilepsy is a common complication of stroke. The number of researchers studying these diseases in both the academic and private sectors is already large and yet growing, providing a long-term driver of potential sales that should be expected to grow over time. In addition, our early results demonstrate that the field currently operates under an incorrect assumption: that the concentration of chloride in the extracellular space just outside the neuronal membrane is set by the surrounding fluid. In reality [Cl⁻]ₒ is on average 50% lower, and varies widely in the extracellular space.

### Definitions

As used herein, the term “about” means “approximately” (e.g., plus or minus approximately 10% of the indicated value).

At various places in the present specification, substituents of compounds of the disclosure are disclosed in groups or in ranges. It is specifically intended that the disclosure include each and every individual subcombination of the members of such groups and ranges.

The term “halo” or “halogen” refers to any radical of fluorine, chlorine, bromine or iodine.

The term “alkyl” refers to a saturated hydrocarbon chain that may be a straight chain or branched chain, containing the indicated number of carbon atoms. For example, C₁-C₆ alkyl indicates that the group may have from 1 to 6 (inclusive) carbon atoms in it. Any atom can be optionally substituted, e.g., by one or more substituents. Examples of alkyl groups include, without limitation, methyl, ethyl, n-propyl, isopropyl, and tert-butyl.

The term “alkenyl” refers to a straight or branched hydrocarbon chain containing the indicated number of carbon atoms and having one or more carbon-carbon double bonds. Any atom can be optionally substituted, e.g., by one or more substituents.

Alkenyl groups can include, e.g., vinyl, allyl, 1-butenyl, and 2-hexenyl. One of the double bond carbons can optionally be the point of attachment of the alkenyl substituent.

The term “cycloalkyl” refers to a fully saturated monocyclic, bicyclic, tricyclic, or other polycyclic hydrocarbon group. Any atom can be optionally substituted, e.g., by one or more substituents. A ring carbon serves as the point of attachment of a cycloalkyl group to another moiety. Cycloalkyl moieties can include groups such as cyclopropyl, cyclobutyl, cyclopentyl, cyclohexyl, cycloheptyl, adamantyl, and norbornyl (bicyclo[2.2.1]heptyl).

The term “heterocyclyl” refers to a fully saturated monocyclic, bicyclic, tricyclic or other polycyclic ring system having one or more constituent heteroatom ring atoms independently selected from O, N (it is understood that one or two additional groups may be present to complete the nitrogen valence and/or form a salt), or S. The heteroatom or ring carbon can be the point of attachment of the heterocyclyl substituent to another moiety. Any atom can be optionally substituted, e.g., by one or more substituents. Heterocyclyl groups can include groups such as tetrahydrofuryl, tetrahydropyranyl, piperidyl (piperidino), piperazinyl, morpholinyl (morpholino), pyrrolinyl, and pyrrolidinyl. By way of example, a phrase such as “heterocyclic ring containing from 5-6 ring atoms”, wherein from 1-2 of the ring atoms is independently selected from N, NH, N(C₁-C₆ alkyl), NC(O)(C₁-C₆ alkyl), O, and S; and wherein said heterocyclic ring is optionally substituted with from 1-3 independently selected Ra would include (but not be limited to) tetrahydrofuryl, tetrahydropyranyl, piperidyl (piperidino), piperazinyl, morpholinyl (morpholino), pyrrolinyl, and pyrrolidinyl.

The term “aryl” refers to an aromatic monocyclic, bicyclic (2 fused rings), tricyclic (3 fused rings), or polycyclic (>3 fused rings) hydrocarbon ring system. One or more ring atoms can be optionally substituted by one or more substituents for example. Aryl moieties include groups such as phenyl and naphthyl.

The term “heteroaryl” refers to an aromatic monocyclic, bicyclic (2 fused rings), tricyclic (3 fused rings), or polycyclic (>3 fused rings) hydrocarbon groups having one or more heteroatom ring atoms independently selected from O, N (it is understood that one or two additional groups may be present to complete the nitrogen valence and/or form a salt), or S. One or more ring atoms can be optionally substituted, e.g., by one or more substituents. Examples of heteroaryl groups include, but are not limited to, 2H-pyrrolyl, 3H-indolyl, 4H-quinolizinyl, acridinyl, benzo[b]thienyl, benzothiazolyl, B3-carbolinyl, carbazolyl, coumarinyl, chromenyl, cinnolinyl, dibenzo[b,d]furanyl, furazanyl, furyl, imidazolyl, imidizolyl, indazolyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, and xanthenyl.

As used herein, the term “haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “heteroalkyl” refers to an alkyl group in which one or more skeletal atoms of the alkyl are selected from an atom other than carbon, e.g., oxygen, nitrogen, sulfur, phosphorus or combinations thereof. In one aspect, a heteroalkyl is a C₁-C₆ heteroalkyl.

As used herein, the term “alkoxy”, employed alone or in combination with other terms, refers to a group of formula —O-alkyl, wherein the alkyl group has n to m carbons.

Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “dextran” refers to a poly(glucose) having an α-(1-6) linked D-glucose main chain with branches from α-1,3 linkages.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

As used herein, the term “subject” refers to any animal, including mammals, preferably mice, rats, other rodents, rabbits, dogs, cats, swine, cattle, sheep, horses, or primates, and most preferably humans.

### EXAMPLES

Materials: Chemicals were purchased from Sigma-Aldrich (St Louis, MO) unless otherwise noted. Solutions are brought up in distilled water unless otherwise noted. Standard aCSF was brought up in 18MQ water and consisted of (in mM): NaCl 126; KCl 3.5; CaCl₂ 2; MgCl₂ 1.3; NaH₂PO₄ 1.2; Glucose 11; NaHCO₃ 15. Low chloride aCSF was identical except that sodium gluconate was substituted for NaCl, resulting in a final Cl⁻ concentration of 10 mM. Calibration solutions were made by mixing appropriate proportions of standard aCSF with low chloride aCSF such that a chloride and gluconate sum total of 136 mM was maintained while Cl⁻ ranged from 10 to 136 mM. Colorimetric assays were read using a Wallac Victor-2 1420 spectrophotometer with a halogen continuous wave light source and spectral line filters at listed wavelength+/−5 to 10 nm. Centrifugation steps were accomplished in a tabletop Eppendorf 5417R microcentrifuge. Reflux apparatus for synthesis consisted of a recirculating cooling bath (Fisher Scientific) filled with ethylene glycol cooling a 24/40 double-lumen coiled reflux tube (Ace Glassware, Vineland, NJ) with a 500 mL round bottom flask (Corning) heated with a heating mantle regulated by a timed power controller (Glas-Col #0406 and #104A PL312, respectively

### Example 1: Synthesis of Conjugates

Dextran-fluorophore conjugates can be synthesized according to Scheme I.

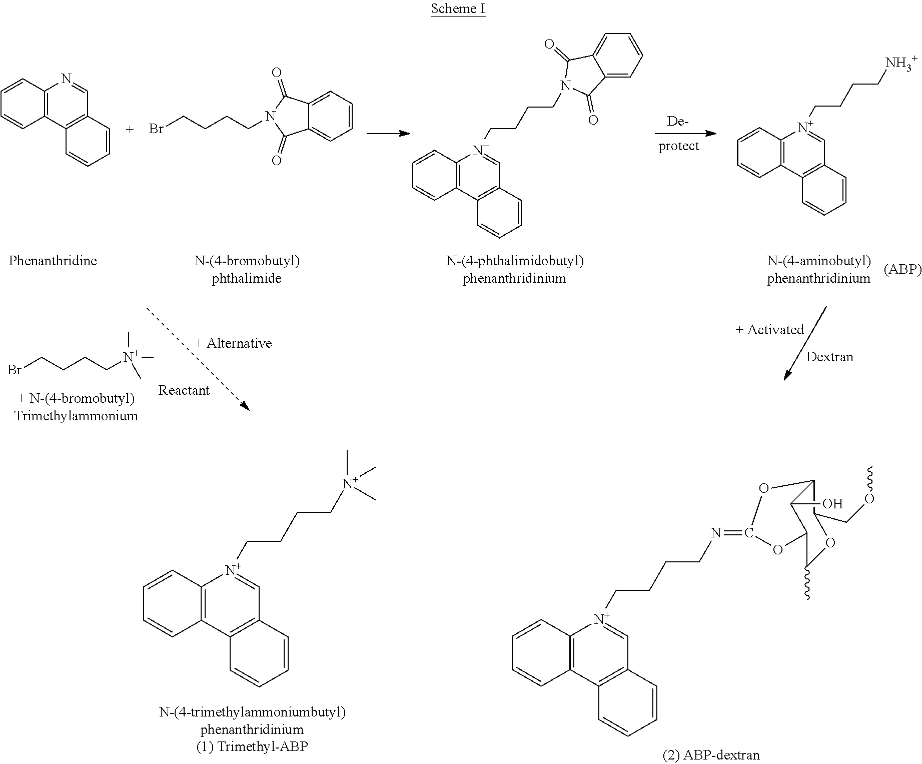

Alkyl chains were added to nitrogen heterocyclic organic backbones using either 1) sulfones of a ring size corresponding to desired alkyl chain length when adding a sulfate group¹¹, or 2) an alkyl chain bearing a reactive bromide on one terminal carbon and on the other terminal carbon either a primary amine protected by a phthalimide group or else a trimethylated ammonium group. The trimethylated moiety was directly recrystallized from 95% ethanol, whereas the primary amine moiety required a deprotection reflux of the phthalimide intermediate with aqueous acid. Bicyclic compounds (quinoline and isoquinoline based) were purified by extraction in the aqueous phase from 4:1 chloroform:water prior to recrystallization from 95% ethanol. Phthalimide intermediates were retained upon filtration with Whatman-40 filter paper from warm reaction mixtures. Deprotected primary amine moieties where filtered through Whatman-40 filter paper while phthalate was retained, and this filtration was repeated at increasingly cold temperatures before recrystallization from 95% ethanol.

### Synthesis of N(4-aminobutyl)phenantridinium (ABP)

N-(4-aminobutyl)phenantridinium (ABP) is a phenanthridine-based aminoalkyl moiety that is suitable for conjugation to dextran. ABP was synthesized in a manner similar to N(3-trimethylaminopropyl)phenantridinium by reflux of equimolar amounts of phenantridine and N(4-bromobutyl)phthalimide in acetonitrile overnight. A pale yellow-green precipitate was separated by filtration through Whatman-40 paper. From this intermediate phthalimide-protected compound the phthalate group was removed by reflux in 6N HCl overnight and subsequent filtration, similar to procedures described for 6-methoxy-N(3-aminopropyl)quilolinium. N(4-aminobutyl)phenantridinium (ABP) was twice recrystallized from hot 95% ethanol/5% deionized water and was stored as its chloride salt in a sealed glass vial protected from light until conjugation to glucose polymer (i.e. dextran).

### Conjugation of ABP to Dextran:

800 mg of 10,000 Dalton (10KDAa) dextran (Millipore-Sigma, D9260) was dissolved in 10 mL distilled water and activated by addition of cyanogen bromide at a 1:4 molar ratio to glucose units within the dextran. Adjustment of pH to 10.7 was accomplished with 10M NaOH initially, then 2M NaOH until 10.7 pH was maintained for >10 minutes without further correction. ABP was added at a 1.1:4 molar ratio to glucose units within the dextran (10% molar excess over calculated binding sites). The pH was adjusted with 2M NaOH to >9.3 and once maintained between 9.3-9.5 pH for >30 minutes without further correction, the mixture was allowed to stir overnight at 4° C. The entire process was protected from light at all steps to as great a degree as possible while maintaining the requisite control over pH. The reaction is then quenched by addition of Tris buffer (0.05 volumes of 1M pH 8.0) and transferred to 3.5KDAa dialysis tubing (SpectraPor 132720) and allowed to dialyze against 50 volumes of distilled water changed twice daily for 4 changes, yielding 30-40 mL of 20-30 mg/mL product. The resultant ABP-dextran is conjugated through its primary amine yielding a tertiary amine bound to one glucose unit within the polymer⁷ at a final molar ratio of 4.4 ABP molecules conjugated to each 10KDAa dextran polymer. It should be noted that the considerable amount of chloride that may have been present in the ABP salt would have been dialyzed out during this process. ABP-dextran was confirmed resilient to both autoclaving and −20° C. freeze-thaw cycles and was thus aliquoted and stored under sterile conditions, protected from light. Once thawed, ABP-dextran could be kept at 4° C. for at least two weeks or if kept under sterile conditions at least six weeks, again protected from light.

### Example 2: In Vitro Studies

Spectrophotometry: Absorption readings were obtained using a Nanodrop One system (Thermo Scientific), while excitation and emission scans were obtained using a Spectramax M2 dual grating system (Molecular Devices). Intensity scans relative to excitation wavelength obtained on either of our custom two-photon excitation (2PE) rigs were analyzed with ImageJ and plotted using Matlab (v2022b).

Fluorescence Lifetime Imaging (FLIM): Time-correlated single-photon counting (TCSPC) FLIM measurements were obtained using one of two custom MaiTai Ti:Sapph laser-scanning two-photon microscopes with high-sensitivity photomultiplier tube (PMT) detectors. The first customized microscope was used for early in vitro experiments and is detailed in the in vitro imaging section below; the second was custom made for in vivo experiments and is detailed in the in vivo imaging section below. In either case, ABP-dextran was excited at 760 nm and emitted photons were subjected to a 445/58 bandpass filter (Chroma) prior to detection using high-sensitivity PMTs. Specific FLIM collection software differed between the two set-ups and is detailed in the relevant sections below, but in either case each pixel had an associated histogram expressing photon number vs photon travel time (the delay between excitation and detection) which was fitted to a single exponential curve to derive a time constant k,

$${A}_{t} = {A}_{o} {e}^{- k t} \qquad ( 1 )$$

denoting the fluorescence lifetime of the fluorophore derived from each pixel. As implied by Equation 1, fluorescence lifetime is an intrinsic property of the fluorophore and wholly independent of fluorophore concentration. However, fluorescence lifetime may be shortened (i.e., quenched) by one or more moieties in a manner dependent upon the concentration of the quencher—not the concentration of the fluorophore itself. In the case of the fluorophore used in the present study (ABP), chloride is the sole biologically relevant quencher. The degree of lifetime shortening relative to the initial unquenched lifetime is linearly related to the concentration of the quencher through the Stern-Volmer relationship,

$$\frac{{t}_{o}}{{t}_{i}} = 1 + [ Q ] {k}_{SV} \qquad ( 2 )$$

where tₒ tᵢ represents the unquenched lifetime divided by the shortened lifetime at a given quencher concentration ([Q]), necessarily yielding¹³ a y-intercept of 1. The slope of the resultant plot, referred to as the Stem-Volmer constant (k^{SV}), facilitates conversion of measured fluorescence lifetime to concentration of quencher. Notably, there is no term to describe fluorophore concentration in Equation 2 as the fluorophore concentration has no impact on measured lifetime. Only the concentration of the quencher, chloride, affects the fluorescence lifetime. Images are acquired over 1.5 minutes (in vivo and agarose gels) to 2.5 minutes (in vitro slice cultures). Each pixel is characterized using a histogram of photons populating time bins from which a single exponential time constant (fluorescence lifetime; the term k in equation 1) and intensity value (summed photons from all bins for that pixel) can be derived.

Calibration of ABP-dextran: ABP-dextran was calibrated against chloride in simple phosphate buffer (20 mM; pH 7.2) with varying amounts of NaCl (0-150 mM), such that solutions were essentially modified PBS. Whether other biologically relevant anions were capable of quenching ABP was verified using similar buffers with 20 mM phosphate and 0-150 mM anion, or 4-(2-hydroxyethyl)-1-piperazineethanesulfonic acid (HEPES) buffer (20 mM; pH 7.2) when varying phosphate concentration. For subsequent experiments done in aCSF or in vivo, ABP-dextran was calibrated against aCSF with varying chloride concentrations (FIG. 4 B&C). This was accomplished by preparing standard aCSF (in mM: NaCl 126; KCl 3.5; CaCl₂) 2; MgCl₂ 1.3; NaH₂PO₄ 1.2; Glucose 11; NaHCO₃ 15) and a low-chloride aCSF in which NaCl was replaced with sodium gluconate, having demonstrated gluconate had no appreciable effect on ABP-dextran quenching (FIG. 7 C). An appropriate amounts of this low chloride aCSF was mixed with standard aCSF to achieve varying chloride concentrations (10-136 mM) in which the sum of sodium chloride and sodium gluconate was kept constant at 136 mM. FLIM calibrations were obtained for each batch of ABP-dextran on the same microscope and with the same range of laser powers used in experiments, though k_{SV} did not appreciably change between ABP-dextran batches. Because calibrations done in aCSF have a minimum Cl⁻ of 10 mM as described, the unquenched lifetime (tₒ) used for Stern-Volmer calculations is approximated as the y-intercept (Cl⁻=0 mM) of a best-fit curve of the raw calibration data; empirically a biexponential fit was used. All calculations are otherwise done as described under Fluorescence Lifetime Imaging (FLIM) section above.

In vitro organotypic slice cultures and imaging: Organotypic hippocampal slice cultures were prepared either as glass-mounted¹⁴ or membrane insert-mounted¹⁵ cultures. Briefly, in either case hippocampi are obtained from P6-8 mice and cut to 400 mm thick slices. These are then gently transferred to a 6-well dish containing either a membrane insert (Millipore) or a poly-L-lysine coated coverslip (Electron Microscopy Sciences), are fed twice weekly with 1 mL neurobasal-A media supplemented with 500 mM Glutamax, 2% B-27, and 0.03 mg/mL gentamycin (all from Invitrogen), and are incubated at 35 C in a 5% CO₂ incubator. Cultures are imaged between DIV 7-21 in aCSF warmed to 33° C. and bubbled with 95% O₂/5% CO₂ perfused at a rate of approximately 100 mL/hour unless otherwise noted. Slices were pretreated with 500 mg/mL ABP-dextran for 1-2 hours prior to perfusing with aCSF containing 136 mM chloride and the same concentration of ABP-dextran. Slices were allowed to equilibrate in perfusate for 20-30 minutes before imaging, equaling at least 2 hours of total exposure to ABP-dextran prior to initiation of imaging. Two-photon images were captured with a 20× water-immersion objective, NA 0.90, on a customized Olympus BX50WI microscope equipped with an 80 MHz Ti:Sapphire MaiTai laser (SpectraPhysics) driven with customized software for microscope operation and Becker & Hickl SPC800 FLIM hardware and software for data collection and initial processing in FLIM mode. Photons must pass through a 445/58 bandpass emission filter before PMT detection (Hamamatsu C6438-01). Lifetime and intensity values for each pixel are generated using SPCImage software (Becker & Hickl) before being exported for use in custom Matlab processing routines.

Image processing in ImageJ and Matlab: Lifetime images are obtained either through Becker&Hickl (GmBH) proprietary software (SPCImage) or using the FLIMJ plug-in as part of ImageJ. Each concentration of Cl⁻ in the calibration curve has a normally distributed set of ABP-dextran lifetime values with a near-constant coefficient of variation (standard deviation divided by mean), except at particularly low Cl⁻ values where the coefficient of variation is greater (FIG. 6). Fluorescence lifetime has a physical upper and lower bound over which its values are meaningful (lifetime in the absence of quencher vs fully quenched fluorophore). Specifically ABP-dextran was found to have lifetime values to be linearly related to Cl⁻ from 0 mM-150 mM through the Stern-Volmer relationship.

Image processing Pixels were excluded (set to black) if ABP lifetimes were in excess of the mean lifetime in 0 mM chloride, or if lifetimes were shorter than twice the standard deviation less than the mean lifetime in 150 mM chloride (calculated from best-fit curve to raw calibration data and average coefficient of variation). Excluding all values outside this calibrated range represents an average exclusion of 0.5-1.5% of pixels obtained using Becker&Hickl FLIM (SPCImage) and 0.1-0.5% of pixels using the custom-built two-photon microscope described above.

Statistics: Statistical analysis was done with Matlab 2022a, with access to statistical toolbox functions. Individual tests used are detailed in figure legends. Corresponding p-values are indicated within figures, in the associated figure legends or both.

Commercially available Cl-sensitive fluorophores were developed and optimized for low millimolar intracellular chloride (FIG. 4). These Cl-sensitive fluorescent molecules are quenched by halides, with different kinetic and photophysical properties. From the original sensors based on a derivatized (6-methoxy)quinoline base (SPQ and MEQ) to lucigenin, the largest and most sensitive Cl⁻ sensor incorporating paired acridine heterocyclics, these early examples share well-described structure-activity relationships. However, they are also optimized for measuring intracellular chloride concentration (Cli), which is typically less than 10 mM and an order of magnitude less than expected extracellular chloride concentration (Clo). These series of structures underlying the commercially available, collisionally quenched fluorescent chloride sensors have sensitivities to Cl⁻ that are well-matched to Cli but not Clo. A probe that is less sensitive to chloride overall would retain more of its dynamic range at higher chloride concentrations and thus would be more sensitive to Cl⁻ at higher chloride concentrations, making such a probe a better match for measurement of Clo (FIG. 4 A).

An asymmetrical heterotricyclic base structure was used to redshift the excitation wavelength into a range that did not excite endogenous autofluorescent compounds. Next, the length of the alkyl chain separating two nitrogens at either end was tuned to modify the chloride sensitivity of the probes. Then the distal nitrogen was modified to a primary amine for conjugation to dextran to make N-(4-aminobutyl)phenantridinium (ABP; FIG. 5 C). One measure of how compatible candidate fluorescent probes are for Clo measurement was to compare the linearized slope of fluorescent lifetime plotted against Cl⁻ over the expected range of Cl⁻ one needs to measure (FIG. 5A, shaded region). Cli is rarely recorded at greater than 40 mM and Clo in brain interstitial space is expected to be equal to that of CSF (110 mM), thus the probes' change of fluorescent lifetime over this range of Cl⁻ was compared (i.e., fluorescence lifetime dynamic range from 40-110 mM Cl⁻; FIG. 5D). Probes optimized for Cli measurement had poor dynamic range at these concentrations, being mostly quenched at Cl⁻ below 40 mM. N-4(sulfobutyl)isoquinolinium (NSBQ) appeared promising, but despite altering properties to make derivative compounds the issue of autofluorescent signal was persistent for this series due to the short excitation wavelength (710 nm using 2PE). Another promising candidate was N-sulfopropylacridinium (SPA), but the expected dimness of the probe combined with its red-shifted emission wavelength that would occupy the blue-green spectral space needed for intracellular chloride-sensitive fluorophores made SPA less useful for our purposes. ABP has appropriate red-shifting of excitation but not emission wavelength, a tuned alkyl chain length to alter sensitivity to Cl⁻ to the desired concentration range, and a primary amine to allow extracellular compartmentalization by conjugation to 10 kilodalton (10KDAa) dextran. Synthesis of ABP was informed by established methods⁷ for synthesizing related compounds 6-methoxy-N-(3-aminopropyl)quinolinium (APQ) and N-(3-trimethylammoniumpropyl)phenanthridinium (TMAPP), modified for our purposes (Scheme 1). Briefly, ABP was synthesized by equimolar reflux of phenanthridine with a phthalimide-protected primary amine-bearing compound (N-(4-bromobutyl)phthalimide) followed by deprotection and subsequent conjugation of the primary amine to dextran activated with cyanogen bromide (see Methods). The related compound with a trimethylated ammonium group that does not allow conjugation to dextran (N-(4-trimethylammoniumbutyl)phenanthridinium) is achieved in a single synthetic step and would be useful if proven to remain in the extracellular space.

Initial characterization of ABP and ABP-dextran: The novel fluorophore was excited at the longest wavelength possible to minimize any autofluorescent signal or tissue damage. ABP-dextran has a bimodal absorbance spectrum (FIG. 5 A, blue line) with a consistent fluorescent excitation profile (FIG. 5 A, black line). Excitation at 360 nm, the longest single-photon excitation wavelength that still achieves maximal excitation was used. Emission spectrum (FIG. 5 A, red line) is in the blue range with a maximum at 420 nm. Using two-photon excitation (2PE) microscopy, the ABP-dextran excitation maximum was determined to be 770 nm (FIG. 5 B), but excitation at 760 nm was used to increase spectral separation from other potential fluorophores at the expense of 10-15% of emission intensity.

The dextran activation was further optimized and conjugation parameters to maximize the apparent number of conjugation sites and the amount of ABP to achieve an optimal labeling ratio of ABP:Dextran (FIG. 5 C). It was found that while intensity initially increased with more ABP added to the reaction mixture, the fluorescence lifetime of ABP did not change appreciably (FIG. 5 C, dark blue and light blue bars, respectively). Once an 8:1 molar ratio of ABP:Dextran in the conjugation reaction was exceeded, the labeling ratio was maximized at approximately 4.4moL ABP to 1moL dextran (FIG. 5 C, annotation above each lifetime/intensity pair). There was no significant difference between the unquenched lifetimes of ABP-dextrans prepared with different labeling ratios (FIG. 5 C, blue bars) nor was there a significant difference between the k_{SV} calculated for these and the k_{SV} calculated for the most recent batches of fully optimized ABP-dextran (FIG. 5 3D).

Optimization of ABP-dextran: With a standardized synthetic process to make ABP-dextran, experimental conditions with a FLIM-capable 2PE microscope using 760 nm excitation and a 445/58 bandpass emission filter were optimized. Empirically around 200 μg/mL ABP-dextran was necessary to obtain adequate signal, thus ABP-dextran concentrations over one order of magnitude from 100-1000 μg/mL (i.e. 10-100 μM) was tested. While intensity increased linearly with ABP-dextran concentration, fluorophore lifetime was consistent but drifted to modestly lower mean values (FIG. 6 A, red and blue lines, respectively). Notably, as ABP-dextran is labeled at a molar ratio of 4.4, 100 M ABP-dextran corresponds to 440 μM ABP. Looking at histograms of raw data from three concentrations of ABP-dextran (FIG. 6 B&C), it can be seen that while intensity varies directly with histogram width (i.e., more signal results in greater variance; FIG. 6 B), lifetime varies inversely with histogram width (i.e., more signal results in less variance; FIG. 6 C). This likely is due to more photons detected in each pixel, yielding more data points from which fluorescent lifetime is calculated in each pixel and decreasing the variance of calculated lifetimes. This results in an asymmetric histogram tightening whereby the right shoulder (longer lifetimes) are lost preferentially over the left shoulder (shorter lifetimes) with minimal alteration of the mode (FIG. 6 C). This asymmetry explains the modest drift to shorter mean lifetimes with greater ABP-dextran concentration observed in FIG. 6 A (blue line). To quantify this variance, the coefficient of variation (standard deviation divided by mean) against ABP-dextran concentration was plotted (FIG. 6 D) and both lifetime and intensity measurements feature a decreasing coefficient of variation and appear to approach a shared asymptote (blue and red lines, respectively). This decrease in coefficient of variance with increasing ABP-dextran concentration is more pronounced in the case of fluorescence lifetime than intensity. As long as ABP-dextran concentration is at least 500 μg/mL (50 μM), the fluorescence lifetime coefficient of variation remains acceptably, if arbitrarily, low (<0.1, arbitrary units). In all future experiments, ABP-dextran at 500 μg/mL unless otherwise noted, was used.

ABP-dextran performance as a Clo probe: ABP-dextran was functionally characterized as a Cl⁻ reporter under experimental conditions anticipated in both in vitro and in vivo settings. Other potential sources of signal instability including batch-to-batch variation, survival after autoclave sterilization, and variation due to pH differences, oxidation, and other biologically relevant anions need to be ruled out.

ABP-dextran was not sensitive to pH over the physiological range (6.8-7.8 pH; FIG. 7 A). Next ABP-dextran solutions was bubbled with nitrogen or oxygen to test for oxidative quenching (FIG. 7 B). While there was an oxygen-dependent quenching effect of statistical significance, this modest effect was considered an acceptably small deviation given that calibration solutions used are similarly oxygenated. Next other biologically relevant anions for ability to quench ABP-dextran were tested. Sodium gluconate was an acceptable inactive substitute for sodium chloride when preparing calibration solutions of equivalent ionic strength. Other biologically relevant anions including phosphate, organic sulfate (methylsulfonate), and bicarbonate, each with measured k_{SV}<1.2M⁻¹ were tested (FIG. 7 C). These alternative anions possessed k_{SV} ranging from 0.11M⁻¹-1.21M⁻¹ versus ABP-dextran k_{SV} for chloride that was found to be 14.7+/−0.5M⁻¹ (mean+/−standard deviation).

The modified dextran confugated was also tested for stability by subjecting it to freeze-thaw (data not shown) and autoclave cycles (FIG. 7 D); neither affected its characteristics as a chloride sensor. Finally, having optimized synthesis of the ABP primary amine, the dextran activation and conjugation steps, and the experimental conditions on two microscopes with different FLIM hardware and software, it ewas found that ABP-dextran performed consistently (FIG. 7 E&F). ABP-dextran FLIM characteristics of consecutive synthetic batches were nearly identical, so that data almost overlay (FIG. 7 E). While microscope to microscope differences are expected, particularly with a technique that relies on measuring photon flight time, the differences in k_{SV} calculated for each of 6 total batches of ABP-dextran was not significantly different between microscopes (FIG. 7 F).

### Example 3: In Vivo Studies

Animals: All animal protocols were approved by the Massachusetts General Hospital Institutional Animal Care and Use Committee. Wild type mice (C57bl/6; Jackson Labs 000664) of either sex were used for this study. Mouse pups remained in the home cage with the dam under standard husbandry conditions until postnatal day 6 to 8 (P6-8) when organotypic slice cultures were prepared or until a cortical window was surgically placed at P26-30.

In vivo murine cortical window imaging: Cortical windows were placed in young adult mice (P26-30) for acute (non-survival) imaging in accordance with Massachusetts General Hospital Institutional Animal Care and Use Committee policies and procedures (protocol 2018N000221). Full methods are included as a supplement. Briefly, anaesthetized mice are immobilized with standard ear bars and nosepiece on a warmed breadboard until a custom headbar is placed. A section of scalp is removed to expose a roughly 1 cm section of skull. Acrylic dental cement powder (Lang Dental, Wheeling IL) mixed with cyanoacrylate adhesive is used to fix the headbar to the exposed skull centered at 2 mm posterior and 3 mm lateral to Bregma, creating a 5 mm diameter working area. A 2.5 mm round section of skull and underlying dura is removed and a 3 mm No. 1 coverslip is placed over the exposed cortex. Coverslip has 10 mL concentrated ABP-dextran/agarose mixture (1% w/v low gelation temperature agarose with 10 mg/mL ABP-dextran in aCSF) pipetted from 42 C heat block immediately before inversion and placement. One hour is allowed before image acquisition for ABP-dextran diffusion into cortex from overlying agarose (between cortex and coverslip; FIG. 5B) during which time the warmed breadboard with immobilization apparatus and anesthetized mouse is transferred to the in vivo imaging microscope, a custom-made gantry-type two-photon microscope equipped with a MaiTai 80 MHz Ti:Sapphire laser (Spectraphysics) and driven with customized ScanImage software (MBF Bioscience, Williston, VT). Photons are detected by a high-sensitivity PMT (Hamamatsu C5594-12) after passing through a 445/58 bandpass emission filter (Chroma) and digitized using custom ScanImage software. All other components are from Thor Labs. The initial analysis of raw FLIM data generating fluorescence lifetime data for each pixel is accomplished using ImageJ plug-in FLIMJ. Subsequent analyses and plot generation are accomplished using custom routines in Matlab. Statistics were generated using relevant Matlab functions (Matlab v2022b, including statistics toolbox).

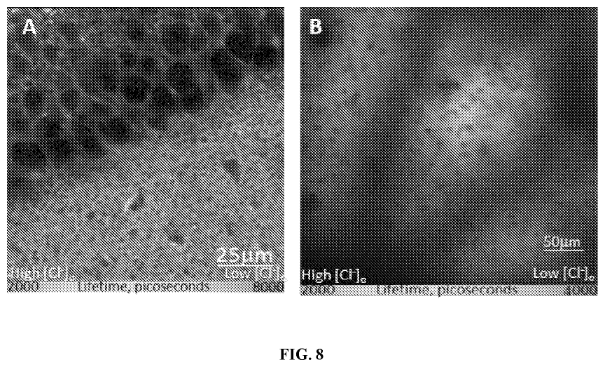

Hippocampal slice cultures (HSCs) of wild-type mice were imaged at DIV7-14 after pretreatment with 500 mg/mM ABP-dextran in aCSF containing 136 mM Cl⁻ (see Methods). The representative image shown in FIG. 8 A details the CA3 pyramidal cell layer and associated stratum radiatum to the lower right, demonstrating silhouettes caused by ABP-dextran exclusion from neuronal cell bodies in stratum pyramidale and larger neurites and small vessels in stratum radiatum (FIG. 8A, upper left and lower right, respectively). HSC was imaged 45 mm below the upper surface of the slice. The upper surface of the slice was determined from the change in fluorescence intensity occurring at the transition from aCSF to brain tissue. This transition was determined at the center of the field of view to account for the natural dome-shaped curvature that HSCs obtain over time. In vivo images obtained through a cortical window implanted in P26-P30 wild-type mice were obtained with a custom 2PE microscope built specifically for in vivo experiments (see Methods). ABP-dextran was delivered in vivo by mixing into agar at 10 mg/mL final concentration. The agar was placed between the exposed cortex and overlying glass coverslip at the time of cortical window placement, allowing ABP-dextran to passively diffuse into the cortex. Based on empirical comparison of ABP-dextran brightness in vivo with in vitro experiments at consistent experimental settings, final concentration of ABP-dextran in mouse cortex is estimated to be between 400-600 mg/mL. ABP-dextran is excluded from vessels and cell bodies in vivo (FIG. 8 B). Wild-type animals are used so as not to introduce another fluorophore in these proof-of-concept experiments, but the fact that ABP-dextran emission occupies the typical blue channel spectrum leaves the majority of spectral space, from green to infrared, open for co-labeling.

Exploring a ratiometric Clo probe: Dextran conjugation afforded us the opportunity to additionally label activated dextran with a Cl-insensitive fluorophore to allow ratiometric Clo determination. Fluorescence lifetime imaging is already independent of probe concentration, but it requires investment in specialized hardware and typically requires long acquisition times. A ratiometric dual-labeled dextran could also provide spatial information independent of Cl⁻ which may allow better definition of physical features such as vessel or cell borders. Efforts have been made previously to achieve such a ratiometric Cl⁻ sensor, but were either unsuccessful⁷ or used incompletely insensitive probes with minimal spectral separation from the Cl-sensitive structure¹⁹. At any rate these previous attempts again focus on measuring low values of Cli and are subject to autoquenching effects. ABP-dextran is optimized for Clo and is resistant to autoquenching at similar concentrations.

### Other Embodiments

Whilst the application has been disclosed in particular embodiments, it will be understood by those skilled in the art that certain substitutions, alterations and/or omissions may be made to the embodiments without departing from the spirit of the disclosure. Accordingly, the foregoing description is meant to be exemplary only, and should not limit the scope of the disclosure. All references, scientific articles, patent publications, and any other documents cited herein are hereby incorporated by reference in their entireties.

1. A novel chloride-sensitive fluorophore composition comprised of a tricyclic phenanthridine base first structure and an alkyl amine second structure appended to nitrogen of the phenanthridine ring as illustrated in FIGS. 2 and 3 of the Specification.

2.-6. (canceled)

7. A method for non-invasively measuring the concentration of chloride in the extracellular space comprised of administering the composition of claim 1 to a cell, tissue or organism and wherein the fluorescence is indicative of said extracellular chloride concentration.

8.-11. (canceled)

12. A general method for rapidly measuring salinity in biological, medical, food production or non-biological systems comprised of the application of one or more of the chloride-sensitive fluorophore compounds, as claimed in claim 1, and wherein the fluorescence is indicative of the amount of salinity in said system.

13. A conjugate of formula F-L-P wherein:
F is a heteroaryl;
L is a linker; and
P is a cap group or a polymer chosen from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative thereof.

14.-15. (canceled)

16. The conjugate of claim 13, wherein the heteroaryl is chosen from indole, isoindole, indolizine, benzofuran, isobenzofuran, benzothiophene, isobenzothiophene, indazole, quinoline, isoquinoline, quinolizine, purine, phthalazine, pteridine, naphthyridine, quinoxaline, quinazoline, cinnoline, benzoxazole, benzothiazole, benzimidazole, chromene, benzoxepine, benzoxazepine, benzoxadiazepine, benzothiepine, benzothiazepine, benzothiadiazepine, benzazepine, benzodiazepine, benzofurazan, benzothiadiazole, benzotriazole, carbazole, beta-carboline, acridine, phenazine, dibenzofuran, xanthene, dibenzothiophene, phenothiazine, phenoxazine, phenoxathiin, thianthrene, phenanthridine, phenanthroline, and perimidine.

17.-18. (canceled)

19. The conjugate of claim 13, wherein the linker is methylene, ethylene, propylene, butylene, or pentalene.

20. The conjugate of claim 13, wherein P is N(R^{p})₃⁺, wherein R^{p} is alkyl.

21. (canceled)

22. The conjugate of claim 13, wherein P is a polymer chosen from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative thereof.

23. The conjugate of claim 22, wherein P has a molecular weight of 1 kDa to 5000 kDa molecular weight.

24.-25. (canceled)

26. The conjugate of claim 22, wherein P is dextran.

27. (canceled)

28. A conjugate of formula (Ia) 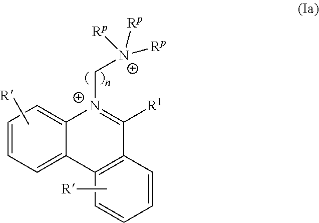
wherein:
each R′ and R¹ is chosen from hydrogen, halogen, alkyl, and heteroalkyl;
n is chosen from 1, 2, 3, 4, 5, and 6; and
each R^{p} independently chosen from alkyl, heteroalkyl, benzyl, and aryl.

29. The conjugate of claim 28, wherein each R′ is hydrogen and R¹ is hydrogen.

30. (canceled)

31. The conjugate of claim 28, wherein n is 3, 4, or 5.

32.-33. (canceled)

34. The conjugate of claim 28, wherein each R^{p} is alkyl.

35. (canceled)

36. A conjugate of formula (Ib) 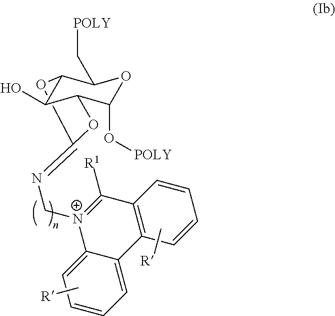
wherein:
each R′ and R¹ is chosen from hydrogen, halogen, alkyl, and heteroalkyl;
n is chosen from 1, 2, 3, 4, 5, and 6; and
each POLY is independently chosen from a dextran polymer.

37. The conjugate of claim 36, wherein each R′ is hydrogen and R¹ is hydrogen.

38. (canceled)

39. The conjugate of claim 36, wherein n is 3, 4, or 5.

40.-41. (canceled)

42. The conjugate of claim 36, wherein the conjugate of formula (Ib) is a chloride-sensitive fluorophore.

43. A pharmaceutical composition comprising a conjugate of claim 36, and a pharmaceutically acceptable excipient.

44. A method for measuring the extracellular chloride concentration in an extracellular space, comprising administering a conjugate of claim 13, and wherein the fluorescence of the compound indicates the extracellular chloride concentration.

45.-52. (canceled)
